(19) United States
(12) Patent Application Publication
(10) Pub. No.: US 2025/0186678 A1
(43) Pub. Date: Jun. 12, 2025

(54) Negative Pressure Medical Device

(71) Applicant: METAL INDUSTRIES RESEARCH & DEVELOPMENT CENTRE, Kaohsiung City (TW)

(72) Inventors: Yur-Ren Kuo, Kaohsiung City (TW); Su-Shin Lee, Kaohsiung City (TW); Chun-Chieh Tseng, Kaohsiung City (TW); Chun-Ming Chen, Kaohsiung City (TW); Tung-Lin Tsai, Tainan City (TW); Chia-Hua Chang, Changhua County (TW); Ling-Zhen Kao, Tainan City (TW)

(21) Appl. No.: 18/530,497

(22) Filed: Dec. 6, 2023

(51) Int. Cl. A61M 1/00

(52) U.S. Cl. CPC A61M 1/966; A61M 2205/3344

(57) ABSTRACT

A negative pressure medical device includes a suction component and a care component. The suction component includes a cylinder body having a chamber. One end of the cylinder body has a suction port connected to the chamber. The other end of the cylinder body opposite the suction port has an insertion port. A plunger rod is disposed in the chamber, such that the plunger rod is movable in a direction along the suction port and the insertion port. The plunger rod abuts an inner peripheral wall of the cylinder body by an airtight component. The care component is configured to cover skin around a wound area of a user to maintain a negative pressure environment between the care component and the wound area. The care component is connected to the suction port of the suction component, such that gas of the negative pressure environment flows unidirectionally to the suction component.

### BACKGROUND OF THE INVENTION

### 1. Field of the Invention

The present invention relates to a medical device and, more particularly, to a negative pressure medical device used to maintain a wound area in a negative pressure state.

### 2. Description of the Related Art

Negative pressure wound therapy (NPWT) is a complementary therapy that involves applying a dressing over a wound to create negative pressure within the space between the dressing and the wound. Research indicates that being in a negative pressure environment contributes to wound healing. Therefore, negative pressure wound therapy is commonly used for healing wounds such as burns, surgical incisions, or traumatic injuries.

In general, the above negative pressure wound therapy is performed by covering the wound area with a layer of dressing to create an airtight space. This airtight space is connected via a suction tube to a powered suction pump. Healthcare professionals can control the powered suction pump to adjust the predetermined suction pressure and suction time, thus generating the intended negative pressure in the airtight space. Due to the need to maintain negative pressure on the wound area for a prolonged period of time, users are required to carry the powered suction pump during the treatment. However, the size of the powered suction pump is bulky and must be carried on a cart or medical rack to assist with carrying. Moreover, the powered suction pump relies on electricity to function, thereby limiting user mobility. Furthermore, the operation of the powered suction pump still relies on healthcare professionals, leading to inconvenience for users in their usage.

### SUMMARY OF THE INVENTION

To solve the above problems, it is an objective of the present invention to provide a negative pressure medical device which is easily portable, thereby provides better convenience in usage.

It is another objective of the present invention to provide a negative pressure medical device which is simple to operate, thereby enabling the user to use it oneself.

As used herein, the term “a”, “an” or “one” for describing the number of the elements and members of the present invention is used for convenience, provides the general meaning of the scope of the present invention, and should be interpreted to include one or at least one. Furthermore, unless explicitly indicated otherwise, the concept of a single component also includes the case of plural components.

As used herein, the term “engagement”, “coupling”, “assembly”, or similar terms is used to include separation of connected members without destroying the members after connection or inseparable connection of the members after connection. A person having ordinary skill in the art would be able to select according to desired demands in the material or assembly of the members to be connected.

A negative pressure medical device according to the present invention includes a suction component and a care component. The suction component includes a cylinder body having a chamber. One end of the cylinder body has a suction port connected to the chamber. The other end of the cylinder body opposite the suction port has an insertion port. A plunger rod is disposed in the chamber, such that the plunger rod is movable in a direction along the suction port and the insertion port. The plunger rod abuts an inner peripheral wall of the cylinder body by an airtight component. The care component is configured to cover skin around a wound area of a user to maintain a negative pressure environment between the care component and the wound area. The care component is connected to the suction port of the suction component, such that gas of the negative pressure environment flows unidirectionally to the suction component.

Therefore, the negative pressure medical device of the present invention draws air from the skin around the wound area by moving the plunger rod in the suction component, thereby creating a negative pressure environment at the skin around the wound area. That is, it is possible to perform negative pressure wound therapy on the skin around the wound area without a power supply. Thus, the user does not need to carry a heavy and bulky powered suction pump, but can easily move for daily life, which can achieve the effect of improving the user's quality of life. Furthermore, by simply controlling the displacement of the plunger rod within the cylinder body, users can independently perform negative pressure wound therapy without the need for healthcare professionals to operate the device. Thus, the effect of reducing the consumption of manpower of healthcare professionals can be achieved.

In an example, one end of the cylinder body is shrunk to form the suction port. Thus, the suction port can be easily coupled to the three-way valve.

In an example, the chamber adjacent to the suction port forms a stopping part by reducing a diameter of the cylinder body. T Thus, the stopping part can be abutted by one end of the plunger rod.

In an example, the airtight component is a plug, and the airtight component is located at one end of the plunger rod facing the suction port. Thus, the plunger rod can drive the airtight component and draw gas through the suction port.

In an example, the airtight component is a ring, and the airtight component is disposed around a circumferential surface of the plunger rod, such that the airtight component radially abuts the inner peripheral wall of the cylinder body. Thus, the plunger rod can drive the airtight component and draw gas through the suction port.

In an example, the negative pressure medical device according to the present invention further includes a three-way valve with an airflow passage. The airflow passage is connected to a first connecting port, a second connecting port and a pressure detection port of the three-way valve. The first connecting port is connected to the suction port of the suction component. The pressure detection port is connected to a pressure meter. The three-way valve includes a switching component for switching the connection between the airflow passage and the first connecting port, the second connecting port, and the pressure detection port, respectively. The care component has an attachment surface and a through hole. An opening at one end of the through hole is located at the attachment surface, and an opening at the other end of the through hole is connected to the second connecting port of the three-way valve. Thus, the user does not need to carry a heavy and bulky powered suction pump, but can easily move for daily life, which can achieve the effect of improving the user's quality of life.

In an example, the airflow passage is a T-shaped channel, such that the first connecting port and the second connecting port are located at opposite ends in an X-direction, and the pressure detection port is located at an end in a Y-direction perpendicular to the X-direction. Thus, the switching component can be positioned at the intersection of the X direction and the Y direction in the airflow passage so that the switching component can sequentially block the connection between the airflow passage and the first connecting port, the second connecting port, and the pressure detection port.

In an example, the through hole of the care component is connected to the second connecting port of the three-way valve by a connecting hose. Thus, the connecting hose can provide a larger range of movement for the suction component, facilitating user operation and enhancing operational convenience.

In an example, the first connecting port of the three-way valve is detachably connected to the suction port of the suction component. Thus, the effect of improving the user's quality of life can be achieved.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 is a front view of a preferred embodiment of the invention.

When the terms “front”, “rear”, “left”, “right”, “up”, “down”, “top”, “bottom”, “inner”, “outer”, “side”, and similar terms are used herein, it should be understood that these terms have reference only to the structure shown in the drawings as it would appear to a person viewing the drawings and are utilized only to facilitate describing the invention, rather than restricting the invention.

### DETAILED DESCRIPTION OF THE INVENTION

In order to make the above and other objectives, features, and advantages of the present invention clearer and easier to understand, preferred embodiments of the present invention will be described hereinafter in connection with the accompanying drawings. Furthermore, the elements designated by the same reference numeral in various FIGURES will be deemed as identical, and the description thereof will be omitted.

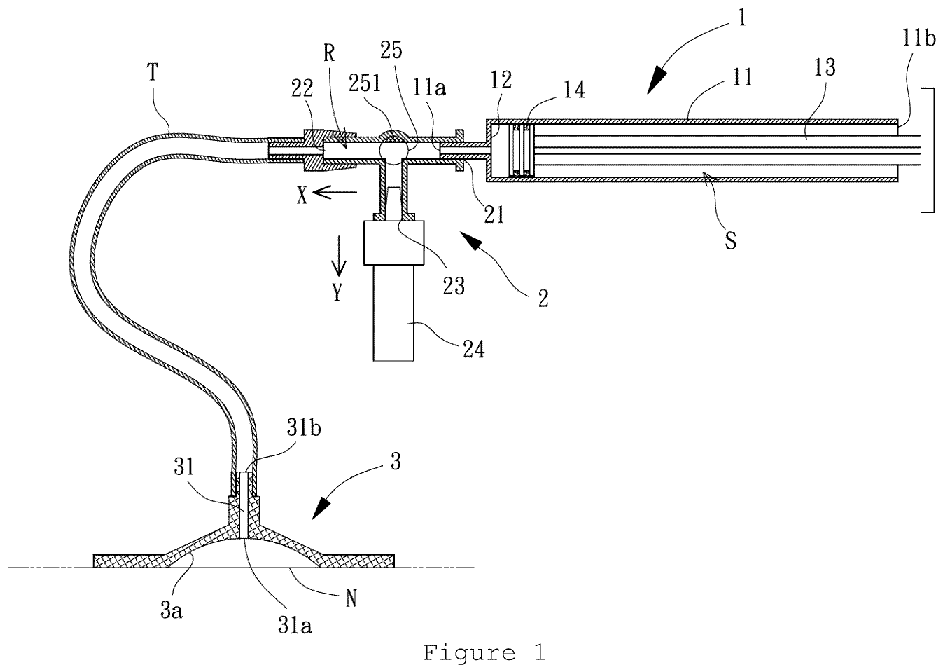

With reference to FIG. 1, which is a preferred embodiment of the negative pressure medical device of the present invention, the negative pressure medical device includes a suction component 1, a three-way valve 2, and a care component 3. The suction component 1 applies suction to the care component 3 via the three-way valve 2.

The suction component 1 includes a cylinder body 11. A chamber S is provided in the cylinder body 11. One end of the cylinder body 11 has a suction port 11a connected to the chamber S. Preferably, the end of the cylinder body 11 can be shrunk to form the suction port 11a. In addition, the chamber S adjacent to the suction port 11a can form a stopping part 12 by reducing the diameter of the cylinder body 11. The other end of the cylinder body 11 opposite to the suction port 11a has an insertion port 11b. A plunger rod 13 can be disposed in the chamber S via the insertion port 11b. The plunger rod 13 is movable within the cylinder body 11 in the direction of the suction port 11a and the insertion port 11b. Also, the stopping part 12 can be abut by one end of the plunger rod 13.

It is noted that the plunger rod 13 includes an airtight component 14 which abuts an inner peripheral wall of the cylinder body 11. In particular, the airtight component 14 may be a flexible material such as rubber, so that the airtight component 14 can abut the inner peripheral wall of the cylinder body 11 by extrusion. Thus, an airtight effect may be achieved between the airtight component 14 and the inner peripheral wall of the cylinder body 11. For example, the airtight component 14 can be a plug, and the airtight component 14 can be located at one end of the plunger rod 13. The plunger rod 13 can drive the airtight component 14 to move. In this embodiment, the airtight component 14 is a ring, and the airtight component 14 can be disposed around the circumferential surface of the plunger rod 13, such that the airtight component 14 can radially abut the inner peripheral wall of the cylinder body 11. Preferably, the airtight component 14 can be disposed around the circumferential surface adjacent to one end of the plunger rod 13. Also, a person skilled in the art can understand that by the elastic deformation of the airtight component 14, even if the airtight component 14 abuts the inner peripheral wall of the cylinder body 11, users can still use the plunger rod 13 to drive the airtight component 14 to move.

The three-way valve 2 has an airflow passage R connected to a first connecting port 21. The first connecting port 21 can be connected to the suction port 11a of the suction component 1. For example, the first connecting port 21 can be detachably and directly connected to the suction port 11a, or the first connecting port 21 can be detachably connected to the suction port 11a through a pipe. The airflow passage R is further connected to a second connecting port 22 and a pressure detection port 23. The pressure detection port 23 can be connected to a pressure meter 24 for detecting the pressure in the airflow passage R. The pressure meter 24 can be a conventional pointer type pressure meter or a digital display type pressure meter. The three-way valve 2 includes a switching component 25 for switching the connection between the airflow passage R and the first connecting port 21, the second connecting port 22, and the pressure detection port 23, respectively.

In this embodiment, the airflow passage R is a T-shaped channel, such that the first connecting port 21 and the second connecting port 22 are located at opposite ends in an X-direction, and the pressure detection port 23 is located at an end in a Y-direction perpendicular to the X-direction. The switching component 25 can be disposed at the intersection of the X direction and the Y direction in the airflow passage R. Thus, the switching component 25 can be rotated by, for example, a wrench or a knob, so that a blocking part 251 of the switching component 25 can sequentially block the connection between the airflow passage R and the first connecting port 21, the second connecting port 22, and the pressure detection port 23. Alternatively, the blocking part 251 can be rotated to a position that forms no blocking to the airflow passage R.

The care component 3 has an attachment surface 3a which can be attached to a skin around the wound area N. The care component 3 can be a flexible material such as silicone, or the care component 3 can be an adhesive film having an adhesive property. Thus, the care component 3 can form an airtight fit with the skin around the wound area N to maintain a negative pressure environment between the care component 3 and the wound area. The care component 3 can be connected to the suction port 11a of the suction component 1, such that the gas of the negative pressure environment flows unidirectionally to the suction component 1. In an embodiment, the care component 3 has a through hole 31. An opening 31a at one end of the through hole 31 is located at the attachment surface 3a, and an opening 31b at the other end of the through hole 31 is connected to the second connecting port 22 of the three-way valve 2. The opening 31b can be directly connected to the second connecting port 22. Alternatively, in this embodiment, the through hole 31 can be connected to the second connecting port 22 by a connecting hose T.

When using the negative pressure medical device of the present invention, the care component 3 and the skin around the wound area N forms an airtight fit, and the airflow passage R of the three-way valve 2 is connected to the first connecting port 21, the second connecting port 22, and the pressure detection port 23. Users may move the plunger rod 13 in a direction away from the suction port 11a. For example, the plunger rod 13 drives the airtight component 14 away from the suction port 11a at one end of the cylinder body 11. Thus, gas between the care component 3 and the skin around the wound area N is drawn into the suction component 1 through the three-way valve 2, thereby creating the negative pressure environment at the skin around the wound area N. Since the passages connecting the care component 3 to the three-way valve 2 are airtight spaces, users can draw gas through the suction component 1 while knowing the pressure of the environment of the skin around the wound area N through the pressure meter 24. When the environment of the skin around the wound area N reaches a intended negative pressure, users can block the connection between the airflow passage R of the three-way valve 2 and the first connecting port 21 to stop drawing gas so as to maintain the negative pressure of the environment of the skin around the wound area N. Also, the second connecting port 22 and the pressure detection port 23 maintain a connection to the environment of the skin around the wound area N. Users can continuously monitor the negative pressure of the environment of the skin around the wound area N by the pressure meter 24 of the pressure detection port 23. In addition, when the negative pressure of the environment of the skin around the wound area N changes, the users can restore the connection between the airflow passage R and the first connecting port 21, and then use the suction component 1 for suction to return to the intended negative pressure.

In view of the forgoing, the negative pressure medical device of the present invention draws air from the skin around the wound area by moving the plunger rod in the suction component, thereby creating a negative pressure environment at the skin around the wound area. That is, it is possible to perform negative pressure wound therapy on the skin around the wound area without a power supply. Thus, the user does not need to carry a heavy and bulky powered suction pump, but can easily move for daily life, which can achieve the effect of improving the user's quality of life. Furthermore, by simply controlling the movement of the plunger rod within the cylinder body, users can independently perform negative pressure wound therapy without the need for healthcare professionals to operate the device. Thus, the effect of reducing the consumption of manpower of healthcare professionals can be achieved.

Although the present invention has been described with respect to the above preferred embodiments, these embodiments are not intended to restrict the present invention. Various changes and modifications on the above embodiments made by any person skilled in the art without departing from the spirit and scope of the present invention are still within the technical category protected by the present invention. Accordingly, the scope of the present invention shall include the literal meaning set forth in the appended claims and all changes which come within the range of equivalency of the claims. Furthermore, in a case that several of the above embodiments can be combined, the present invention includes the implementation of any combination.

What is claimed is:

1. A negative pressure medical device, comprising:
a suction component including a cylinder body having a chamber, wherein one end of the cylinder body has a suction port connected to the chamber, and the other end of the cylinder body opposite the suction port has an insertion port, wherein a plunger rod is disposed in the chamber, such that the plunger rod is movable in a direction along the suction port and the insertion port, and wherein the plunger rod abuts an inner peripheral wall of the cylinder body by an airtight component; and
a care component configured to cover skin around a wound area of a user to maintain a negative pressure environment between the care component and the wound area, wherein the care component is connected to the suction port of the suction component, such that gas of the negative pressure environment flows unidirectionally to the suction component.

2. The negative pressure medical device as claimed in claim 1, wherein one end of the cylinder body is shrunk to form the suction port.

3. The negative pressure medical device as claimed in claim 2, wherein the chamber adjacent to the suction port forms a stopping part by reducing a diameter of the cylinder body.

4. The negative pressure medical device as claimed in claim 1, wherein the airtight component is a plug, and wherein the airtight component is located at one end of the plunger rod facing the suction port.

5. The negative pressure medical device as claimed in claim 1, wherein the airtight component is a ring, and wherein the airtight component is disposed around a circumferential surface of the plunger rod, such that the airtight component radially abuts the inner peripheral wall of the cylinder body.

6. The negative pressure medical device as claimed in claim 1, further comprising a three-way valve with an airflow passage, wherein the airflow passage is connected to a first connecting port, a second connecting port and a pressure detection port of the three-way valve, wherein the first connecting port is connected to the suction port of the suction component, wherein the pressure detection port is connected to a pressure meter, wherein the three-way valve includes a switching component for switching the connection between the airflow passage and the first connecting port, the second connecting port, and the pressure detection port, respectively, wherein the care component has an attachment surface and a through hole, and wherein an opening at one end of the through hole is located at the attachment surface, and an opening at the other end of the through hole is connected to the second connecting port of the three-way valve.

7. The negative pressure medical device as claimed in claim 6, wherein the airflow passage is a T-shaped channel, such that the first connecting port and the second connecting port are located at opposite ends in an X-direction, and the pressure detection port is located at an end in a Y-direction perpendicular to the X-direction.

8. The negative pressure medical device as claimed in claim 6, wherein the through hole of the care component is connected to the second connecting port of the three-way valve by a connecting hose.

9. The negative pressure medical device as claimed in claim 6, wherein the first connecting port of the three-way valve is detachably connected to the suction port of the suction component.
